(19) United States
(12) Patent Application Publication
(10) Pub. No.: US 2025/0064165 A1
(43) Pub. Date: Feb. 27, 2025

(54) Footwear Integrated Hazard Avoidance and Fall Detection System

(71) Applicants: Ahmad Salameh Abusukhon, Madaba (JO); Mohammad Khalil Mohammad Nofal, Kiel (DE)

(72) Inventors: Ahmad Salameh Abusukhon, Madaba (JO); Mohammad Khalil Mohammad Nofal, Kiel (DE)

(21) Appl. No.: 18/815,583

(22) Filed: Aug. 26, 2024

Related U.S. Application Data
(60) Provisional application No. 63/578,548, filed on Aug. 24, 2023.

(51) Int. Cl. A43B 3/44; A61B 5/00; A61B 5/103; A61B 5/11

(52) U.S. Cl. CPC A43B 3/44; A61B 5/1036; A61B 5/1117; A61B 5/6807

(57) ABSTRACT

A footwear integrated hazard avoidance and fall detection system having a shoe containing a plurality of sensors, microcontrollers, and a geo-positioning device. The plurality of sensors, in combination with a machine learning algorithm, determine the presence of a true fall event. The data gathered by the plurality of sensors is processed to determine whether the shoes are being worn by a user, and if a true fall event has occurred. Additionally, the system detects the presence of hazards and initiate a response system to mitigate injury to the user.

### FIELD OF THE INVENTION

The present invention relates generally to a system to detect falls. More specifically, the present invention is a footwear integrated system that detects falls, obstacles and hazards, and responds accordingly, while using machine learning algorithms and data processing to differentiate between true fall events and false positives.

### BACKGROUND OF THE INVENTION

The purpose of the invention is to develop a sophisticated smart shoe system capable of detecting user falls, identifying obstacles and hazards, and alerting users the same. The system not only provides data to general users but may also extend its use cases to disabled individuals, elderly populations, diabetics, and professionals such as firefighters. The incorporation of multiple sensors into shoes and a machine learning system distinguishes provides a utility not presented in the prior art by offering an advanced means of identifying true fall events and potential dangers.

For disabled users, especially those with limited mobility, falls can lead to serious injuries and prolonged recovery periods. The smart shoe system addresses this concern by providing real-time fall detection and immediate alerts, ensuring prompt medical assistance. Furthermore, the system's capacity to detect obstacles proves valuable in enhancing navigation for visually impaired users, as well as aiding those with mobility challenges. The functionality of this smart shoe system is not limited to the general public, however, but rather it extends to professionals in high-risk environments. Firefighters, for example, often operate in hazardous conditions with limited visibility and thus the shoe's ability to identify fire hazards enhances their situational awareness, enabling quicker response times and improved safety measures. Additionally, the system may be used to identify a true fall when a firefighter falls and becomes unconscious while he is saving other people.

Existing fall detection systems often rely on wearable devices that cannot differentiate between a true positive and a false positive, referred to herein as true falls and false falls, respectively. Additionally, some systems within the prior art lack accuracy, leading to a high rate of false-falls alarms, which reduces user trust and adoption. Moreover, many solutions overlook the critical aspect of identifying fire hazards or obstacles, leaving users vulnerable to potentially life-threatening situations. Also, the prior art systems do not implement response systems (e.g. protecting the user body using airbags system when falls is recorded) into the shoes but rather only provide data to a user.

The current technologies also fail to cater to specific user groups like firefighters, who require specialized tools to address their unique needs. Conventional footwear, even with added sensors, does not offer the comprehensive functionalities of the proposed smart shoe system.

The present invention may also be useful for powerline workers. In a situation wherein a powerline worker is wearing the shoes while working on elevated electrical powerlines, if he falls, then the present invention can monitor the change in the altitude during the fall using the GPS that is affixed to his shoes. Based on the change in the altitude value, the microcontroller in the first step sends a command to the airbags to be filled with air, thus reducing the risk of bone fractures. In the second step, the system sends a message to the caretaker including the user's location and the level of possible harm.

Additionally, the prior art does not classify the level of harm caused by a fall wherein the present invention is able to do so using the Global Positioning System (GPS) by measuring the altitude during the user fall, and classifying the fall based on the altitude value. Furthermore, the prior art does not disclose a means for protection to protect the user from various hazards during falls. However, the present invention is able to do so by providing a set of airbags that are affixed to various parts of the user body and controlled using a microcontroller. These bags are filled with air during a fall and thus prevent bone fractures. If a fall occurs in water, these bags prevent the user from drowning risks. In addition, no prior art systems investigated the integration of the functionality of the light sensor, the load scale sensor and the vibration sensor in determining if the user is wearing his shoes or not.

An objective of the present invention is to accurately detect true fall events while minimizing false positives. The system's integrated array of sensors works collaboratively to distinguish between normal activities and genuine falls, preventing unnecessary alarms and protecting the user body using a set of airbags. An additional objective is to enable users to navigate through obstacles and hazards efficiently. By employing a combination of depth perception, temperature sensors, and fire detection mechanisms, the system not only identifies physical barriers but also identifies fire risks, enhancing overall safety. The integration of multiple sensors facilitates the collection of comprehensive data, contributing to accurate fall detection, hazard identification, and environmental awareness. Further, a machine learning system employs pattern recognition and anomaly detection algorithms, enhancing the system's ability to distinguish between false positives and true fall events.

In conclusion, the proposed smart shoe system represents an innovative advancement in fall detection, hazard warning, body protection (using airbags system) and user assistance (with the help of the GPS system affixed to the user shoes). By addressing the limitations of existing solutions and catering to the needs of disabled individuals and professionals alike, this invention promises to enhance safety, mobility, and situational awareness in a variety of contexts. Through the integration of advanced sensors, machine learning capabilities, and user-friendly design, the smart shoe system introduces a comprehensive and reliable solution to the challenges faced by users in both everyday scenarios and specialized professions.

### SUMMARY OF THE INVENTION

A footwear integrated system for detecting falls, avoiding hazards, and responding to events (e.g. protecting the user body using airbags system when a fall occurs, responds to drowning risks, and determine the user location using a GPS system) having a system of sensors to determine various metrics pertaining to the status of the user. The system comprises multiple sensors that work collaboratively to determine whether the shoe is being worn by the user as a means to prevent a false positive reading (i.e. false falls). Additionally, a user worn inflation device, water detection sensors, and geographic positioning systems allow for the system to respond to drowning risks. A mobile application in addition to the footwear allows for communication with a cloud network to process data, analyze data using machine learning systems, and retrieve data in real time.

### BRIEF DESCRIPTION OF THE DRAWINGS

FIG. 1 is a diagram of the system of the present invention.

FIG. 2 is a chart of the components of the shoes of the present invention.

FIG. 3 is a chart of the plurality of sensors of the present invention.

FIG. 4 is a chart of the hardware of the present invention.

FIG. 5 is a diagram of the upper portion of the pair of shoes and the corresponding sensors of the present invention.

FIG. 6 is a diagram of the sole of the present invention showing the sensors of the present invention.

FIG. 7 is a diagram of the interior of the pair of shoes showing the light sensor of the present invention.

FIG. 8 is a chart of the user mobile device of the present invention.

FIG. 9 is a chart of the cloud network of the present invention.

FIG. 10 is a diagram of the system of the present invention.

FIG. 11 is a chart of the user worn flotation device of the present invention.

FIG. 12 is a diagram of the user worn flotation device of the present invention.

FIG. 13 is a process diagram of the true fall detection and response system of the present invention.

FIG. 14 is a process diagram of the determination of a true fall event.

FIG. 15 is a process diagram of the obstacle detection and avoidance system of the present invention.

FIG. 16 is a process diagram of the high temperature hazard detection and avoidance system of the present invention.

FIG. 17 is a process diagram of the drowning risk detection and response system of the present invention.

FIG. 18 is a process diagram of the system determining if the shoes are being worn by the user.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a footwear integrated hazard avoidance and fall detection system, embodiments of the present disclosure are not limited to use only in this context.

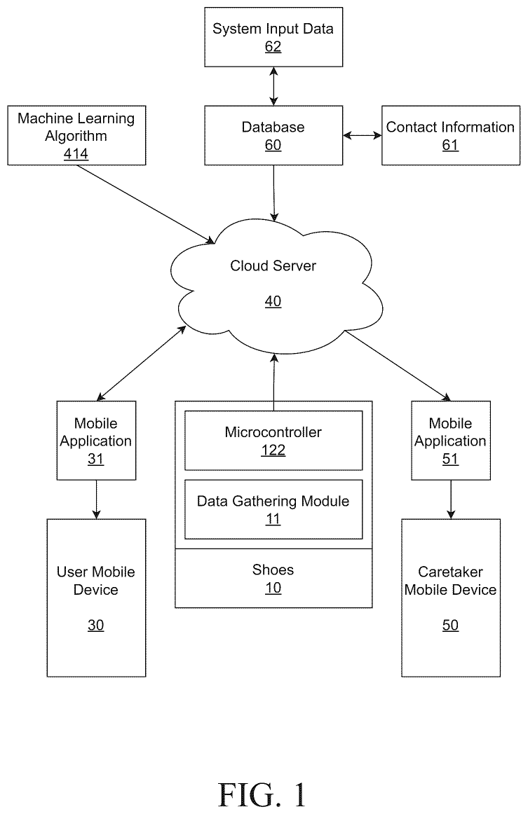

As shown in FIG. 1 through FIG. 15, the present invention is a footwear integrated hazard avoidance and fall detection system comprising an at least one shoe 10, an at least one mobile device 30, and a cloud network 40. In the preferred embodiment of the present invention, as shown in FIG. 1, the at least one shoe 10 is a pair of shoes, comprising a left shoe and a right shoe wherein said left and right shoes are mirrored versions of each other. Furthermore, in the preferred embodiment of the present invention, the at least one mobile device 30 comprises a plurality of mobile devices 30,50 wherein such embodiments, the plurality of mobile devices 30,50 comprises a first mobile device 30 and a second mobile device 50. In the preferred embodiments of the present invention, the first mobile device 30 is a device 30 possessed by a user wherein said user is a first party user whereby said user wears the at least one shoe 10. Moreover, in the preferred embodiment of the present invention, the second mobile device 50 is a third-party mobile device wherein said third party may be a caretaker. The first mobile device 30 and second mobile device 50 are also referred to herein as the user device 30 and the caretaker device 50, respectively.

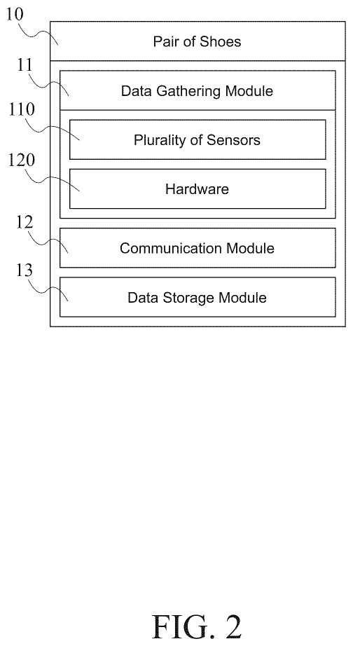

As shown in FIG. 2, the pair of shoes 10 comprises a data gathering module 11, a communication module 12, and a data storage module 13. In the preferred embodiment of the present invention, the data gathering module 11 comprises a plurality of sensors 110 and a hardware system 120. In the context of the present invention, the data storage module 13 stores data collected by the plurality of sensors 110 onto the cloud network 40. Additionally, within the context of the present invention, the communication module 12 communicates data gathered from the plurality of sensors 110. In the preferred embodiment of the present invention, the communication module 12 communicates and transfers data gathered by the plurality of the sensors 110 to the cloud network 40.

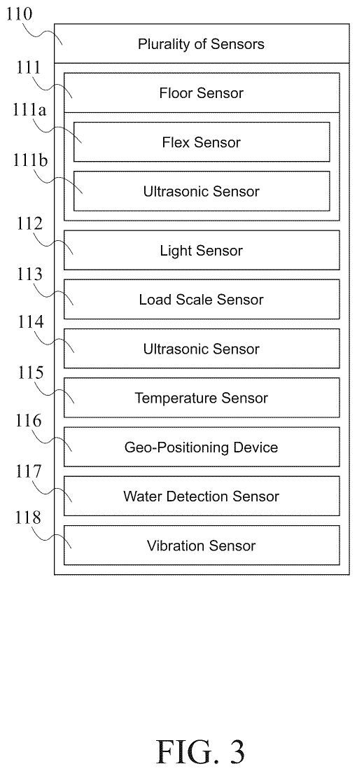

As shown in FIG. 3, in the preferred embodiment of the present invention, the plurality of sensors 110 comprises a plurality of floor sensors 111, a light sensor 112, a load scale sensor 113, an ultrasonic sensor 114, a temperature sensor 115, a geo-positioning device 116, a water detection sensor 117, and a vibration sensor 118. In the context of some embodiments of the present invention, the floor sensor 111 may detect the presence of the ground surface by detecting the ground surface within a predetermined threshold of the floor sensor 111. In some embodiments, the floor sensor 111 may detect the presence of a ground surface by making contact with the ground surface. In the preferred embodiment of the present invention, the plurality of floor sensors 111 comprises at least one of: a flex sensor 111a and an ultrasonic sensor 111b. In the context of the present invention, a flex sensor 111a is a resistive sensor that measures the amount of bending or flexing in a particular direction, wherein the flex sensor 111a is composed of a flexible substrate, such as plastic or polymer, coated with a conductive material, whereby the resistance across the sensor changes proportionally to the degree of bending. Referring to the flex sensor 111a, the change in resistance may be measured and translated into an electrical signal, which is used to determine the angle or degree of flexion. In the context of the present invention, a light sensor 112 is an electronic device that detects and measures the intensity, presence, or variation of light within a specific wavelength range, such as visible, infrared, or ultraviolet light whereby the light sensor 112 converts the detected light into an electrical signal, which can be utilized to trigger actions in the present invention. In the preferred embodiments of the present invention, the light sensor 112 comprises photodiodes, photoresistors, and phototransistors. In the context of the present invention, an ultrasonic sensor 114 is a device that uses ultrasonic sound waves to detect the presence, distance, and movement of objects, obstacles, and ground surfaces. In the preferred embodiment of the present invention, the ultrasonic sensor 114 emits a sound wave and measures the time for the wave to reflect off an object and return to the ultrasonic sensor 114, thereby determining the distance to the object. In the context of the present invention, a temperature sensor 115 is an electronic device that measures the temperature of the proximate environment and converts the data into an electrical signal, such as a thermocouple, a resistance temperature detector (RTDs), a thermistor, and a semiconductor-based sensor. Further, in the context of the present invention, a geo-positioning device 116, including at least one of either a GPS (Global Positioning System) and a GSM (Global System for Mobile Communications) device, determines and transmits the geographic location of the device. Moreover, in the context of the present invention, a water detection sensor 117 is a device designed to detect the presence of water or moisture in an environment. In the context of the present invention, a load scale sensor 113 is an electronic device that measures the load on the load sensor which converts said load exerted on the load sensor 113 into an electrical signal that is converted into a value in kilograms. Lastly, in the context of the present invention, a vibration sensor 118, also referred to herein as a vibration transducer, is a device that detects vibrations or oscillations in the present invention and converts them into an electrical signal. In some embodiments of the present invention, the vibration sensor 118 may be a piezoelectric device, an accelerometer, or a capacitive device.

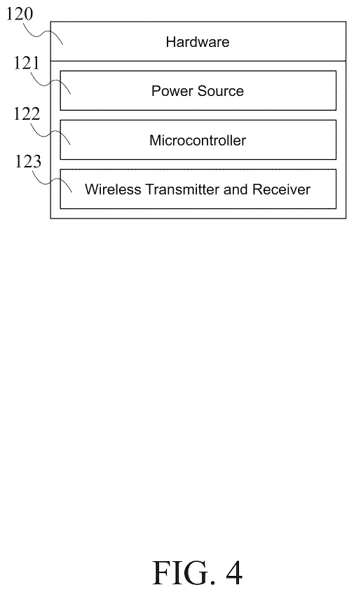

Additionally, as shown in FIG. 4, the hardware system comprises a power source 121, a microcontroller 122, and a wireless communication device 123. In the preferred embodiment of the present invention, the power source 121 may comprise a battery. Additionally, in the preferred embodiment of the present invention, the microcontroller 122, also referred to herein as a microprocessor and micro-processing unit, is an integrated circuit which performs computer executable methods comprising a central processing unit (CPU), a memory (both volatile and non-volatile), an at least one input/output (I/O) peripheral and a set of digital and analog pins. As understood, the microcontroller 122 executes a set of programmed instructions to in regard to the present invention.

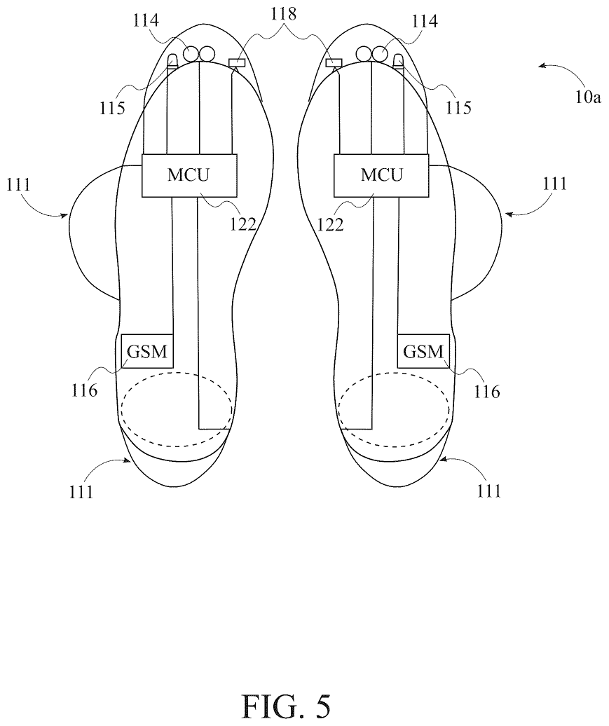

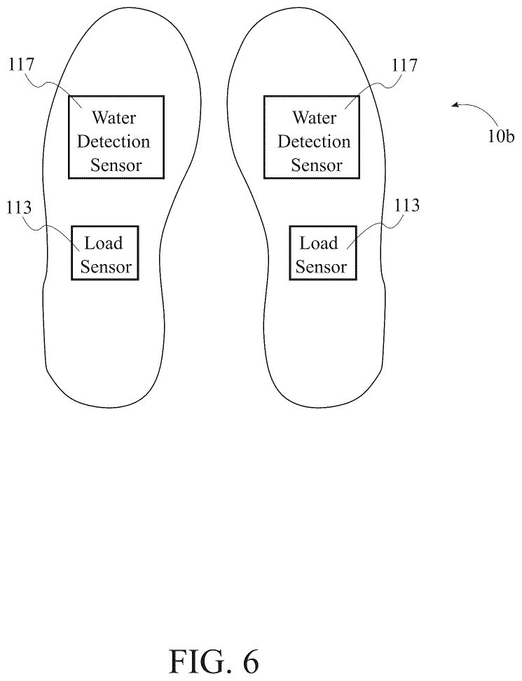

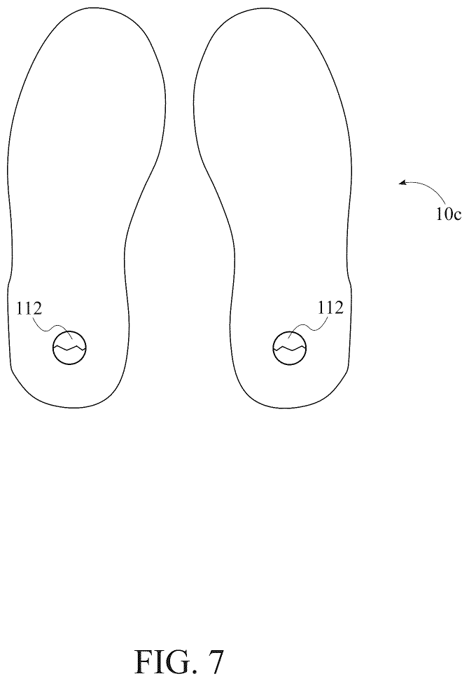

As shown in FIG. 5, the plurality of floor sensors 111, the ultrasonic sensor 114, the temperature sensor 115, the geo-positioning device 116, the vibration sensor 118, and the microcontroller 122 are positioned on an upper portion 10a of the shoe 10. In the context of the present invention, the upper portion 10a of the shoe 10 is the portion of the shoe covering the top of the user's foot, wherein the aforementioned plurality of sensors 111,114,115,116,118 and the microcontroller 122 are positioned on the exterior outwardly surface of the upper portion 10a of the shoe 10. In the preferred embodiment, the plurality of floor sensors 111 are positioned on a front-most portion of the shoe 10, a rear-most portion of the shoe, and an outwardly lateral portion of the shoe. In such embodiments, the floor sensors 111 are configured in a manner to detect a near proximity of a ground surface, relative to the portions of the shoe that would only be proximate the ground surface in an event wherein the user has fallen, referred to herein as a true fall. Additionally, in the preferred embodiment of the present invention, the ultrasonic sensor 114, the temperature sensor 115, and the vibration sensor 118 are positioned on the forward-most portion of the shoe (the toe). In the preferred embodiment, the ultrasonic sensor 114, the temperature sensor 115, and the vibration sensor 118 are configured in a manner to collect data from the front of the user (i.e. the direction of motion of the user). As shown in FIG. 6, the water detection sensor 117 and the load sensor 113 are configured on a sole 10b of the shoe 10. Furthermore, as shown in FIG. 7, the light sensor 112 is positioned within an interior portion 10c of the shoe 10. In the preferred embodiment of the present invention, the light sensor 112 is configured within the interior portion 10c of the shoe 10, proximate the opening of the shoe 10c. In the preferred embodiment of the present invention, the light sensor 112 is positioned in a manner whereby the light is undetectable when the user is wearing the shoe 10, thus indicating to the system that the user is wearing the shoe 10, based on the data collected by the light sensor 112.

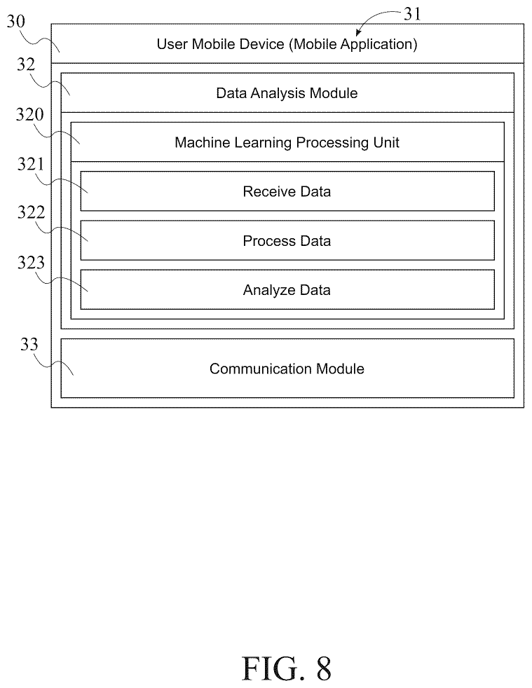

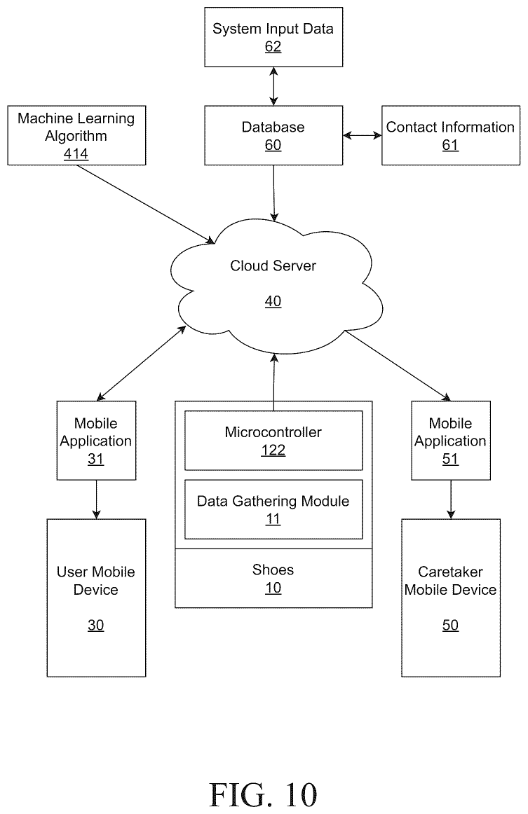

As shown in FIG. 8, in the preferred embodiment of the present invention, the user mobile device 30, comprises a computer executable method 31, also referred to herein as a mobile application 31 and mobile app. Within the context of the present invention, a mobile application 31 is a software program which runs on the mobile devices 30,50 of the present invention and executes a programmable computer executable method to facilitate operations of the present invention. In the preferred embodiment of the present invention, each of the plurality of mobile devices 30,50 comprise a mobile application 31,51. As shown in FIG. 8, the mobile application 31 comprises a data analysis module 32 wherein said data analysis module 32 receives 321 and processes 322 data gathered from the plurality of sensors 110. Additionally, in the preferred embodiment of the present invention, the mobile application 31 comprises a communication module 33 wherein said communication module 33 provides two-way communication between the mobile device 30 and adjacent systems including the cloud network 40. Furthermore, in the preferred embodiment of the present invention, the data analysis module 32 comprises a machine learning processing unit 320 wherein the machine learning processing unit 320 receives 321 the data from the plurality of sensors 110, processes the data 322 into machine readable format, and analyzes 323 the data according to machine learning algorithms 414 (FIG. 10). In the preferred embodiment of the present invention, the machine learning processing unit 320 is trained through supervised learning, unsupervised learning, and reinforcement learning techniques using a collection of data gathered by the plurality of sensors 110, whereby data inputs pertaining to a true fall event is identified and used to train the machine learning model. In the preferred embodiment of the present invention, the system detects and responds to the presence of a true fall event. A true fall event, in the context of the present invention, is an event whereby the system registered a fall and the user wearing the shoes 10 falls. To determine the presence of a true fall event, the machine learning algorithm 414 processes 322 the input data from the sensors 110 to determine if the pair of shoes 10 is being worn by the user by measuring the input data against a predetermined threshold for each of the plurality of sensors including the light sensor 112, the vibration sensor 118, and the load scale sensor 113.

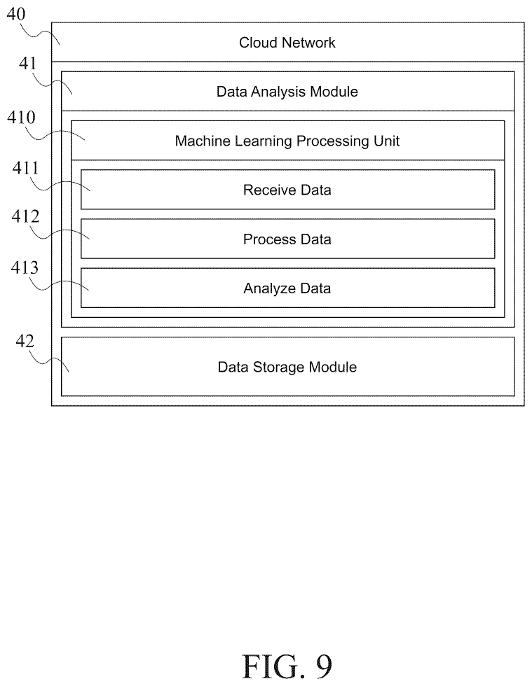

As shown in FIG. 9, the cloud network 40 comprises a data analysis module 41 wherein said data analysis module 41 comprises a machine learning processing unit 410 wherein the machine learning processing unit 410 receives 411 the data from the plurality of sensors 110, processes 412 the data into machine readable format, and analyzes 413 the data according to machine learning algorithms 414 (FIG. 10). Furthermore, as shown in FIG. 10, in the preferred embodiment of the present invention, the cloud network 40 further comprises a data storage module 42 (FIG. 9) wherein said data storage module 42 comprises a data-base 60 comprising system input data 62 and user contact information 61.

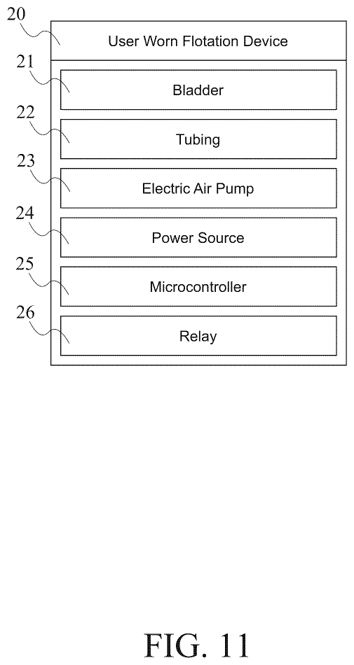

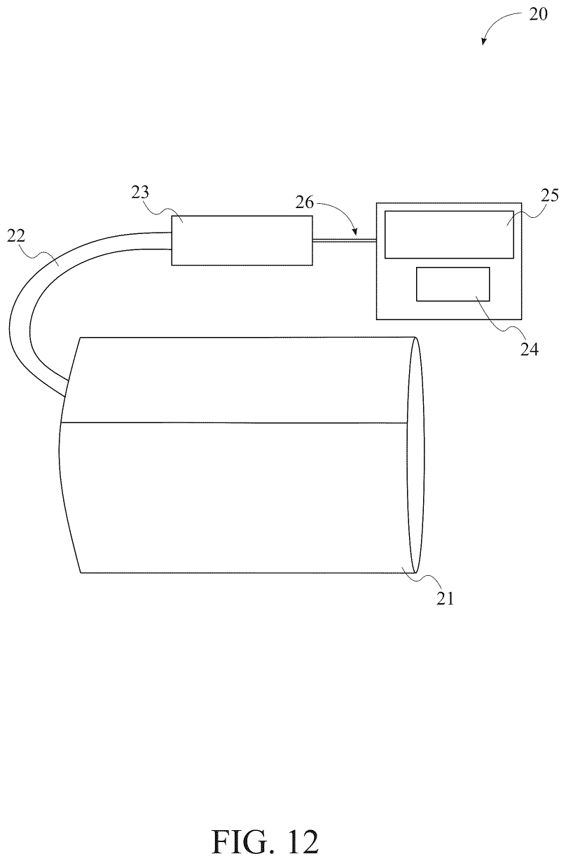

As shown in FIG. 11 and FIG. 12, in some embodiments of the present invention, the footwear integrated hazard avoidance and fall detection system 1 further comprises a user worn flotation device 20, also referred to herein as a user worn inflation device 20. In the preferred embodiment of the present invention, the user worn inflation device 20 comprises a bladder 21, a section of tubing 22, an air pump 23, a power source 24, a microcontroller 25, and a relay 26. The bladder 21 is connected to the air pump 23 through the section of tubing 22. The electric air pump 23 is connected to the power source 24 and the microcontroller 25 whereby the microcontroller 25 comprises a relay 26. In the preferred embodiment, the microcontroller controls the relay 26 wherein said relay 26 switched the pump 23 on and off. In the preferred embodiment of the present invention, the air pump 23 forces air into the bladder 21, thereby causing the bladder 21 to inflate. In the preferred embodiment of the present invention, the microcontroller 25 receives data signals from the communication module 12.

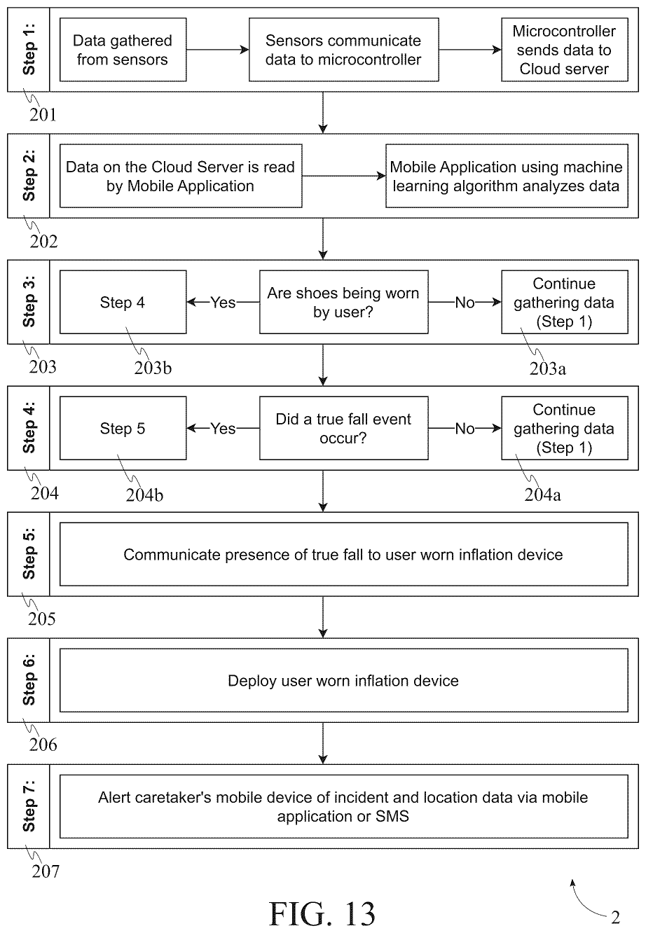

As shown in FIG. 13, the present invention comprises a process for detecting and responding to a true fall 2 comprising a plurality of steps. In a first step 201, data is gathered from the plurality of sensors 110. The sensors 110 communicate the data to the microcontroller 122 wherein the microcontroller 122 then transmits the data to the cloud network 40. In a second step 202, the data that has been transmitted to the cloud network 40 is then accessed and read by the mobile application 31. The mobile application 31 uses the machine learning algorithm 414 to analyze the data. Next, in a third step 203, the machine learning algorithm 414 and the mobile application 31 determine if the shoes 10 are being worn by the user. The machine learning algorithm 414 compares the data collected from the plurality of sensors 110 to predetermined minimum thresholds wherein all of the predetermined thresholds must be met for the algorithm 414 to determine that the shoes 10 are being worn by the user. In the preferred embodiment of the present invention, the light sensor 112, the load scale sensor 113, and the vibration sensor 118 contribute to input data in the determination of whether the shoes 10 are being worn. In the preferred embodiment of the present invention, the threshold for the load scale sensor 113 is 50 kilograms. If the shoes 10 are being worn by the user 203b, in a fourth step 204, given that the system 2 determines that the shoes 10 are being worn, the machine learning algorithm 414 analyzes the data provided by the plurality of sensors 110 including the plurality of floor sensors 111 and the location data from the geographic positioning device 116 to compare the input data from said sensors 111,116 to a set of predetermined thresholds. If the thresholds are met 204b, the algorithm 414 determines that a true fall occurred and a subsequent fifth step 205 is initiated. In the fifth step 205, the presence of the true fall event to the microcontroller 122 and the user worn inflation device 20. Following the communication to the microcontroller 122 and the user worn inflation device 20, a sixth step 206 is initiated wherein said step 206, the user worn inflation device 20 is deployed. In the preferred embodiment, upon determining that the user is in an environment having a risk of true fall, the electric air pump 23 fills the bladder 21 of the inflation device 20, thus protecting the user from the risk of injury. Next, in a seventh step 207, an alert is sent to the caretaker mobile 50 device informing the caretaker of the true fall event of the user and the location of the user. In some embodiments, the alert is sent via short messaging service (SMS) where the message contains the location data of the user. In alternate embodiments of the present invention, the alert is sent via a notification through the mobile application 51.

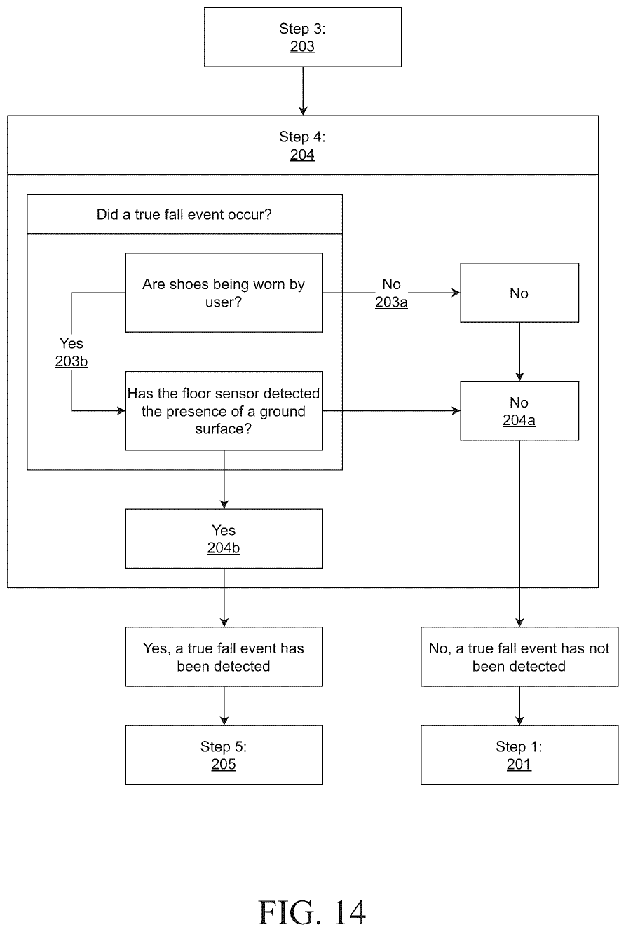

In the fourth step 204 in the process of determining a true fall event 2, as shown in FIG. 14, upon determining whether the shoes 10 are being worn, the system processes the data gathered by the plurality floor sensors 111 to determine the presence of a ground surface. If the machine learning algorithm 414 determines, upon processing the data gathered by the plurality of floor sensors 111, determines that the plurality of floor sensors have detected a ground surface 204b, then the system establishes that a true fall event has occurred and triggers the subsequent fifth step 205. Otherwise, if the system does not determine that the plurality of floor sensors have detected a ground surface 204a, then the process 2 will reiterate, continuing to gather data.

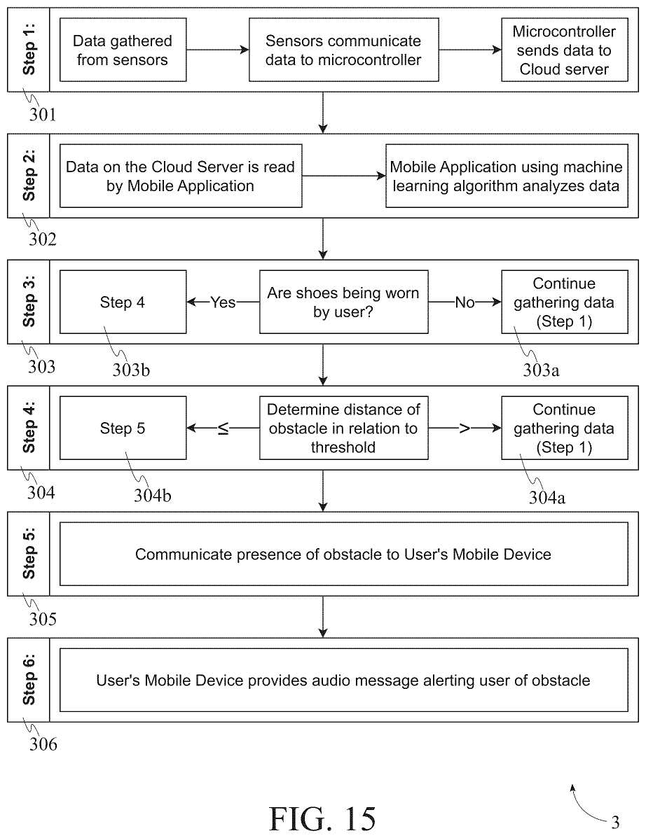

As shown in FIG. 15, the present invention comprises a process for detecting and informing the user of an obstacle hazard 3 comprising a plurality of steps. In a first step 301, data is gathered from the plurality of sensors 110. The sensors 110 communicate the data to the microcontroller 122 wherein the microcontroller 122 then transmits the data to the cloud network 40. In a second step 302, the data that has been transmitted to the cloud network 40 is then accessed and read by the mobile application 31. The mobile application 31 uses the machine learning algorithm 414 to analyze the data. Next, in a third step 303, the machine learning algorithm 414 and the mobile application 31 determine if the shoes 10 are being worn by the user. The machine learning algorithm 414 compares the data collected from the sensors 110 to predetermined minimum thresholds wherein all of the predetermined thresholds must be met for the algorithm 414 to determine that the shoes 10 are being worn by the user. In the preferred embodiment of the present invention, the light sensor 112, the load scale sensor 113, and the vibration sensor 118 contribute to input data in the determination of whether the shoes 10 are being worn. In the preferred embodiment of the present invention, the threshold for the load scale sensor 113 is 50 kilograms. If the shoes are being worn 303b, in a fourth step, the mobile application 31 analyzes the data provided by the ultrasonic sensor 114 to compare the input data from said sensor 114 to a predetermined threshold. If the threshold is met, the mobile application 31 determines that an obstacle is within the path of motion of the user 304b and a subsequent fifth step 305 is initiated. In the preferred embodiment, the threshold for the distance between the ultrasonic sensor 114 and the obstacle is 30 centimeters. Thus, in the preferred embodiment, if the obstacle is within less than or equal to 30 centimeters, then the fifth step 305 will initiate wherein, data communicating the presence of the obstacle is delivered to the user mobile device 30. Next, in a sixth step 306, the user mobile device 30 provides an audio message informing the user of the approaching obstacle.

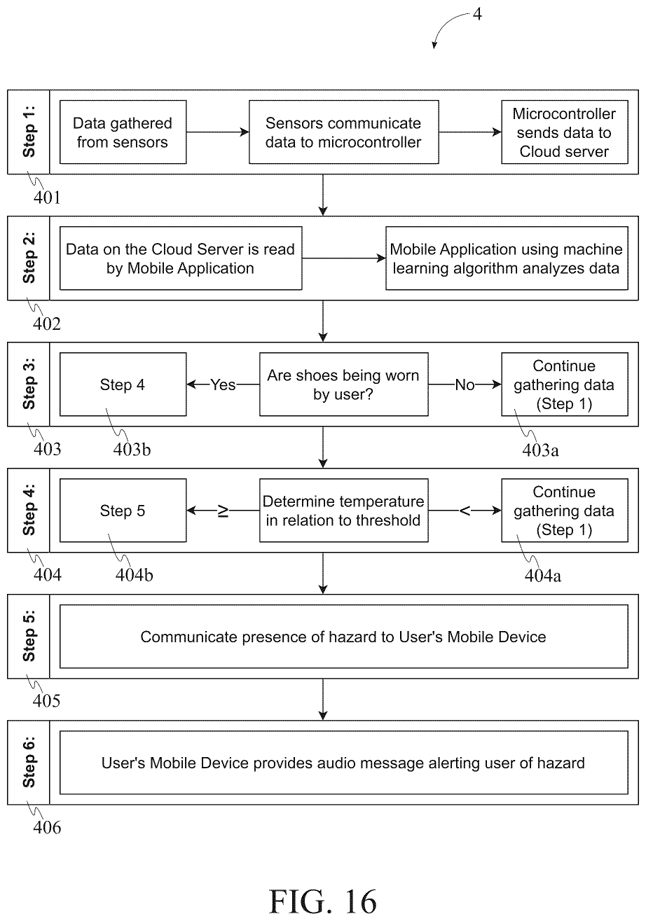

As shown in FIG. 16, the present invention comprises a process for detecting and informing the user of a high temperature hazard 4 comprising a plurality of steps. In the context of the present invention, the high temperature hazard may include a fire exceeding a temperature threshold. In a first step 401, data is gathered from the plurality of sensors 110. The sensors 110 communicate the data to the microcontroller 122 wherein the microcontroller 122 then transmits the data to the cloud network 40. In a second step 402, the data that has been transmitted to the cloud network 40 is then accessed and read by the mobile application 31. The mobile application 31 uses the machine learning algorithm 414 to analyze the data. Next, in a third step 403, the machine learning algorithm 414 and the mobile application 31 determine if the shoes 10 are being worn by the user. The machine learning algorithm 414 compares the data collected from the sensors 110 to predetermined minimum thresholds wherein all of the predetermined thresholds must be met for the algorithm 414 to determine that the shoes 10 are being worn by the user. In the preferred embodiment of the present invention, the light sensor 112, the load scale sensor 113, and the vibration sensor 118 contribute to input data in the determination of whether the shoes 10 are being worn. In the preferred embodiment of the present invention, the threshold for the load scale sensor is 50 kilograms. In a fourth step 404, given that the system determines that the shoes are being worn 403b, the mobile application 31 analyzes the data provided by the temperature sensor 115 to compare the input data from said sensor to a predetermined threshold. If the threshold is met, the algorithm 414 determines that a fire hazard is within the path of motion of the user 404b and a subsequent fifth step 405 is initiated. In the preferred embodiment, the threshold for the temperature reading is 40° C. Thus, in the preferred embodiment, if temperature is greater than or equal to 40° C., then the fifth step 405 will initiate. In the fifth step 405, data communicating the presence of the hazard is delivered to the user mobile device 30. Next, in a sixth step 406, the user mobile device 30 provides an audio message informing the user of the approaching hazard.

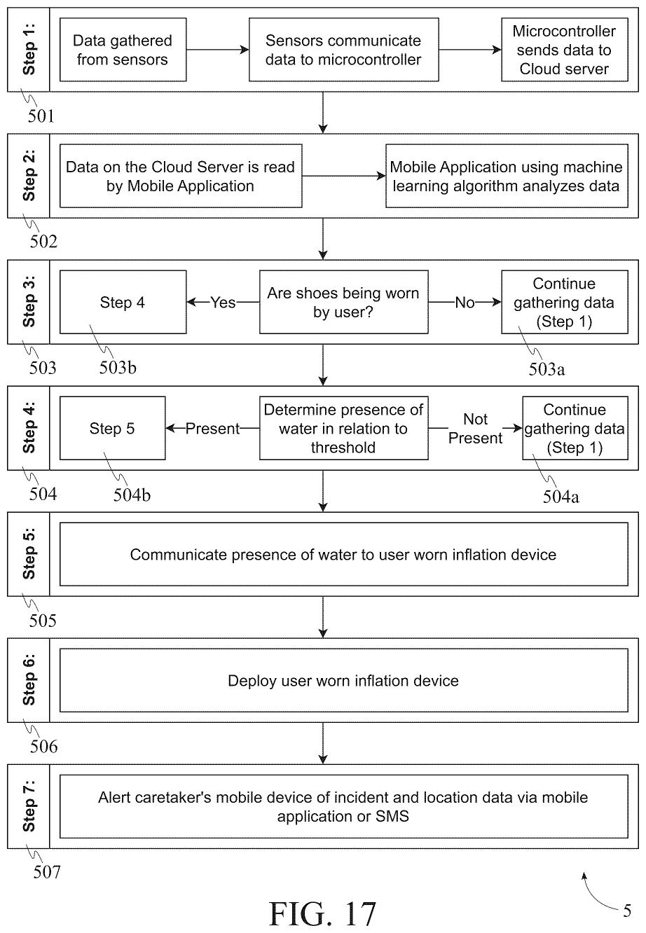

As shown in FIG. 17, the present invention comprises a process for detecting and responding to a potential drowning risk (i.e. a water hazard) 5 comprising a plurality of steps. In a first step 501, data is gathered from the plurality of sensors 110. The sensors 110 communicate the data to the microcontroller 122 wherein the microcontroller 122 then transmits the data to the cloud network 40. In a second step 502, the data that has been transmitted to the cloud network 40 is then accessed and read by the mobile application 31. The mobile application 31 uses the machine learning algorithm 414 to analyze the data. Next, in a third step 503, the machine learning algorithm 414 and the mobile application 31 determine if the shoes 10 are being worn by the user. The machine learning algorithm 414 compares the data collected from the sensors 110 to predetermined minimum thresholds wherein all of the predetermined thresholds must be met for the algorithm 414 to determine that the shoes 10 are being worn by the user. In the preferred embodiment of the present invention, the light sensor 112, the load scale sensor 113, and the vibration sensor 118 contribute to input data in the determination of whether the shoes 10 are being worn. In the preferred embodiment of the present invention, the threshold for the load scale sensor 113 is 50 kilograms. Next, in a fourth step 504, given that the system determines that the shoes are being worn 503b, the machine learning algorithm 414 analyzes the data provided by the plurality of sensors 110, specifically, the water detection sensor 117 and the location data from the geographic positioning device 116 to compare the input data from said sensors 116,117 to a set of predetermined thresholds. If the thresholds are met, the algorithm 414 determines that a user is likely in a hazardous environment wherein the risk of drowning is highly present 504b, and a subsequent fifth step 505 is initiated. The fifth step 505 comprises the communication of data to the microcontroller 122 of the user worn inflation device 20 indicating the drowning risk. Once the data indicating a drowning risk is received, a sixth step 506 initiates wherein the deployment of the user worn inflation device 20 occurs. In the preferred embodiment, upon determining that the user is in an environment having a risk of drowning, the electric air pump 23 fills the bladder 21 of the inflation device 20, thus providing additional buoyancy to the user. In the preferred embodiment, the inflation device 20 is especially functional in the event that the user was rendered unconscious during the fall. In a seventh step 507, an alert is sent to the caretaker mobile device 50 informing the caretaker of the location of the user and the potential threat that exists. In some embodiments, the alert is sent via short messaging service (SMS). In alternate embodiments of the present invention, the alert is sent via a notification through the mobile application 51. In additional embodiments of the present invention, a voice alert notifies the user of a hazard event.

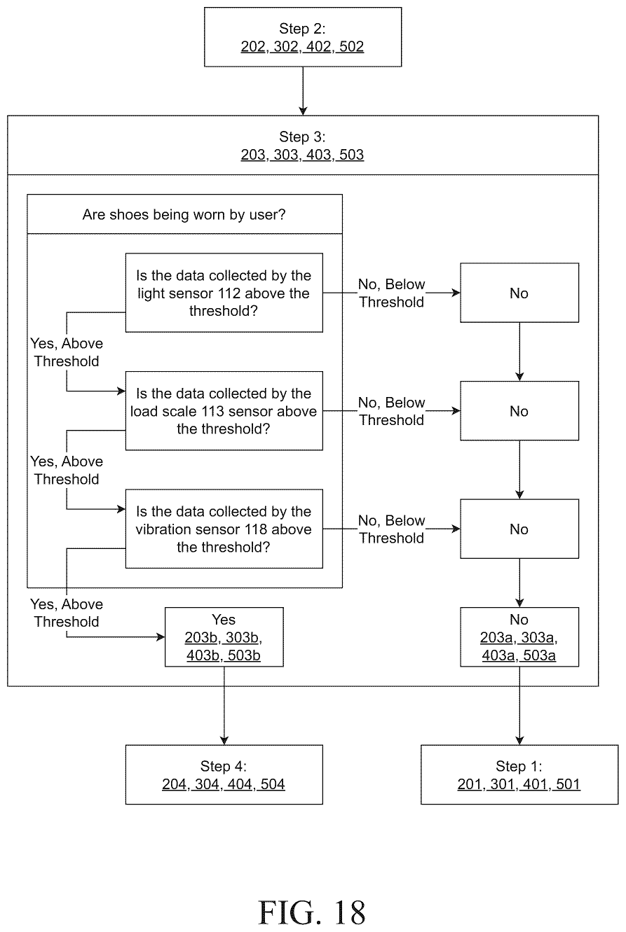

As shown in FIG. 18 in the preferred embodiment of the present invention, during the determination of whether the shoes are being worn by the user in the third steps of the aforementioned processes 203, 303, 403, 503, data gathered from the light sensor 112, the load scale sensor 113, and the vibration sensor 118 is analyzed to determine if the shoes are being worn. If the data gathered by each of the light sensor 112, the load scale sensor 113, and the vibration sensor 118 is determined to be above a predetermined threshold, then the machine learning algorithm 414 will determine that the shoes 10 are being worn by the user. In the event that any of the data gathered by each of the light sensor 112, the load scale sensor 113, and the vibration sensor 118 is determined to be below a predetermined threshold, the machine learning algorithm 414 will determine that the user is not wearing the shoes 10. If the machine learning algorithm 414 determines that the shoes 10 are being worn, then the subsequent step is initiated. Contrarily, if the machine learning algorithm 414 determines that the shoes 10 are not being worn, the system will continue to gather information.

Although the invention has been explained in relation to its preferred embodiment, it is to be understood that many other possible modifications and variations can be made without departing from the spirit and scope of the invention.

What is claimed is:

1. A footwear integrated hazard avoidance and fall detection system comprising:
a shoe comprising:
a data gathering module;
a data storage module;
a communication module; and
a microprocessor; and
a first mobile device comprising:
a data analysis module;
a communication module
wherein:
the data gathering module comprises a plurality of sensors wherein said plurality of sensors comprises a light sensor, a load sensor, and a temperature sensor
the communication module of the shoe comprising a wireless communication device wherein said wireless communication device is a data transmitter and receiver;
the plurality of sensors communicate data to the microcontroller; and
the microcontroller communicates data collected by the plurality of sensors to a cloud network;
the first mobile device comprises a computer executable method by which said data retrieved from the cloud network and processed.

2. The footwear integrated hazard avoidance and fall detection system, as claimed in claim 1, wherein the light sensor is positioned within an interior portion of the shoe.

3. The footwear integrated hazard avoidance and fall detection system, as claimed in claim 1, further comprising a plurality of floor sensor wherein said floor sensors gathers data to determine the presence of a ground surface.

4. The footwear integrated hazard avoidance and fall detection system, as claimed in claim 3, wherein the plurality of floor sensors are flex sensors.

5. The footwear integrated hazard avoidance and fall detection system, as claimed in claim 3, wherein the plurality of floor sensors are ultrasonic sensors.

6. The footwear integrated hazard avoidance and fall detection system, as claimed in claim 3, wherein the data gathering module further comprises an ultrasonic sensor, a geo-positioning device, a water detection sensor, and a vibration sensor.

7. The footwear integrated hazard avoidance and fall detection system, as claimed in claim 1, wherein the shoe comprises:
an upper portion;
a sole;
the interior portion;
wherein:
the upper portion of the shoe comprises a plurality of floor sensors, the temperature sensor, a ultrasonic sensor, a geo-positioning device, and a vibration sensor; and
the sole comprising a water detection sensor and the load sensor.

8. The footwear integrated hazard avoidance and fall detection system, as claimed in claim 1, further comprising a user worn inflation device, wherein said user worn flotation device comprises:
a bladder;
an air pump; and
a microcontroller.

9. The footwear integrated hazard avoidance and fall detection system, as claimed in claim 8, wherein a determination of a true fall event is detected, whereby:
the data gathered by the plurality of sensors is communicated to the microprocessor of the shoe;
the microprocessor of the shoe communicates the data gathered by the plurality of sensors to a cloud server;
the data communicated to the cloud server is received and read by the computer executable method; and
the computer executable method further comprising a machine learning algorithm wherein said machine learning algorithm analyzes the data communicated to the cloud server.

10. The footwear integrated hazard avoidance and fall detection system, as claimed in claim 1, wherein a determination of a true fall event is detected, whereby:
data gathered by the light sensor, the load sensor, and a vibration sensor is processed to determine if the shoe is being worn by a user; and
the light sensor is positioned within the interior portion of the shoe.

11. The footwear integrated hazard avoidance and fall detection system, as claimed in claim 10, whereby:
data gathered by a plurality of floor sensors, in combination with the data gathered by the light sensor, the load sensor, and the vibration sensor is processed to determine if a true fall event occurred.

12. The footwear integrated hazard avoidance and fall detection system, as claimed in claim 10, whereby:
data gathered by a water detection sensor, in combination with the data gathered by the light sensor, the load sensor, and the vibration sensor is processed to determine if a water hazard event is present.

13. The footwear integrated hazard avoidance and fall detection system, as claimed in claim 10, whereby:
data gathered by the temperature sensor, in combination with the data gathered by the light sensor, the load sensor, and the vibration sensor, is processed to determine if a temperature hazard event is present.

14. The footwear integrated hazard avoidance and fall detection system, as claimed in claim 8, wherein:
upon determination of a true fall event, the air pump forces air into the bladder.

15. The footwear integrated hazard avoidance and fall detection system, as claimed in claim 8, whereby:
data gathered by the water detection sensor, in combination within the data gathered by the light sensor, the load sensor, and a vibration sensor, is processed to determine if a water hazard event is present; and
upon the presence of a water hazard event, the air pump forces air into the bladder, thus inflating the user worn inflation device.

16. The footwear integrated hazard avoidance and fall detection system, as claimed in claim 1, wherein the plurality of sensors further comprises a water detection sensor, a vibration sensor, and a floor sensor;
the floor sensor determining the presence of a ground surface;
wherein:
data gathered by the light sensor, the load sensor, and the vibration sensor in combination with data gathered by at least one of the water detection sensor, the floor sensor, and the temperature sensor is processed to determine at least one of the following events including:
a water hazard;
a temperature hazard; and
a true fall event;
upon determination of the at least one event, an alert is sent to at least one of:
the first mobile device belonging to the user; and
a second mobile device wherein said second device is a mobile device belonging to a third party.

17. A footwear integrated hazard avoidance and fall detection system comprising:
a shoe comprising a microcontroller;
a user worn inflation device;
a first mobile device; and
a cloud network;
wherein:
the shoe comprises a data gathering module;
the first mobile device comprises a computer executable method;
the cloud network comprises a set of machine learning algorithms and a database;
the data gathering module collects data through a plurality of sensors;
the data collected by the plurality of sensors is communicated to the cloud network; and
the microcontroller communicates the data to the cloud server.

18. The footwear integrated hazard avoidance and fall detection system, as claimed in claim 17 further comprising:
a second mobile device comprising a computer executable method;
the cloud alerts the second mobile device of at least one of the following including:
a geo-location data collected by the plurality of sensors;
an indication of a true fall event; and
an indication of a water hazard.

19. The footwear integrated hazard avoidance and fall detection system as claimed in claim 17, wherein the plurality of sensors comprises:
a floor sensor;
a light sensor;
a load scale sensor;
an ultrasonic sensor;
a temperature sensor;
a geo-positioning device;
a water detection sensor; and
a vibration sensor.

20. A footwear integrated hazard avoidance and fall detection system comprising:
a shoe comprising:
an upper portion;
an interior portion; and
a sole;
wherein:
the upper portion of the shoe comprises:
a vibration sensor;
a plurality of floor sensors;
an ultrasonic sensor;
a temperature sensor;
the interior portion of the shoe comprises a light sensor; and
the sole comprises a load sensor and a water detection sensor.
